(19) United States
(12) Patent Application Publication
(10) Pub. No.: US 2012/0265210 A1
(43) Pub. Date: Oct. 18, 2012

(54) Vertebroplasty Curved Needle

(76) Inventors: Alexander Grinberg, Raynham, MA (US); Paul S. Maguire, Raynham, MA (US)

(21) Appl. No.: 13/087,632

(22) Filed: Apr. 15, 2011

(51) Int. Cl. A61B 17/58

(52) U.S. Cl. 606/93

(57) ABSTRACT

Devices, systems and methods for delivering a curable, stabilizing material into a central region of a bone structure. Precise placement of the curable substance into a central region of a bone structure through a unipedicular approach. One primary advantage is its ability to create a central cavity in a vertebral body (or to centrally deliver bone cement) through a unipedicular approach. A curved needle having increased rigidity in its flexible end when the end is disposed in its straight position. This increased rigidity is due to a novel tensioning mechanism and to a segmented tube design (as opposed to the conventional longitudinally-slotted tube design).

### BACKGROUND OF THE INVENTION

In vertebroplasty, the surgeon seeks to treat a compression fracture of a vertebral body by injecting bone cement such as PMMA into the fracture site. One clinical report describes mixing two PMMA precursor components (one powder and one liquid) in a dish to produce a viscous bone cement; filling 10 cc syringes with this cement, injecting it into smaller 1 cc syringes, and finally delivering the mixture into the desired area of the vertebral body through needles attached to the smaller syringes.

This injection of the stabilizing material into damaged or compromised bone sites has proven highly beneficial for patients. However, these materials are typically delivered through a straight needle that accesses the vertebral body through a pedicle. Because the pedicle are present at the lateral edges of the vertebral body, pedicle-based delivery has difficulty in delivering material to the central (mid-line) region of the vertebral body. One proposed solution is to fill the central region from a lateral needle tip—however, this approach may lead to overfilling and leakage. Another proposed solution is bipedicular delivery—which is delivery through each of the pedicles. However, the proposed bipedicular access and delivery techniques necessitate multiple needle sticks and therefore a greater risk of tissue damage and infection. Also, neither proposed solution provides precision in the placement of the stabilizing material, which is desirable to prevent overfilling.

Curved needle devices have been proposed as a solution to this issue, but these are prone to breaking due to a lack of strength, rigidity and/or fatigue strength.

Therefore, a need exists in the field of vertebral body augmentation for an improved device for delivering stabilizing material to the damaged or compromised bone sites.

U.S. Pat. No. 5,002,543 (Bradshaw) discloses a steerable tip fracture reduction device. In particular, Bradshaw discloses a steerable intramedullary fracture reduction device has an elongated shaft with a steerable tip pivotally mounted to the distal end of the shaft. A tip actuating apparatus near the proximal end of the shaft enable the operator to steer the tip and the shaft into successive segments of the fractured bone, even when the segments are transversely or rotationally displaced so that the segments can be aligned by the shaft.

U.S. Pat. No. 7,476,226 (Weikel) discloses tools for use in the creation of cavities in bones. The tools include a probe, a cannula that provides percutaneous passageway to the interior of the treated bone, a bone tamp, and a system for delivering bone filler material into the cavity. The bone tamp has a shaft that is inserted into the bone through the cannula. The end of the shaft that is inserted into the bone may have a flapper tip that extends out of axial alignment with the shaft upon deployment by the physician. Once the tip is deployed, the bone tamp can be rotated to form the cavity. The cavity may then be treated with a medicament, filled with bone filler material, or both. Other tools and materials described herein may be used to lift or restore the treated bone closer to its natural anatomy.

US Patent Publication 2002-0026197 (Foley) discloses instrumentation for treatment of the spine, including an elongate member having a deformable distal end portion at least partially formed of a flexible and preferably elastic material. The distal end portion has an initial configuration for placement adjacent a vertebral body and a deformed configuration defining at least one outwardly extending projection for displacement of at least a portion of the vertebral body. The elongate member preferably comprises a rod member, a sleeve member and an actuator mechanism for imparting relative linear displacement between the rod and sleeve members to effect outward deformation of the distal end portion of the sleeve member. In one embodiment, the instrumentation is used to compact cancellous bone to form a cavity within a vertebral body. In another embodiment, the instrumentation is used to reduce a compression fracture. In yet another embodiment, the instrumentation is used to distract a disc space between adjacent vertebral bodies.

US Patent Publication 2010-0010298 (Bakos) discloses an apparatus, system, and method for use with an endoscope. A flexible overtube having a proximal end and a distal end defines a hollow lumen therebetween to receive a flexible shaft portion of an endoscope therein. The proximal end of the flexible overtube is configured to remain outside of a patient and the distal end is configured to enter the patient through a natural orifice. At least one fluid tight seal is located at the proximal end of the flexible overtube to prevent leakage of fluids around the flexible shaft of the endoscope when the flexible shaft of the endoscope is positioned within the flexible overtube. The system further includes a flexible endoscope. The method includes introducing the system into a patient though a natural orifice of the patient and performing an endoscopic translumenal procedure.

### SUMMARY OF THE INVENTION

The present invention relates to devices and methods for stabilizing bone structures. More particularly, it relates to devices, systems and methods for delivering a curable, stabilizing material into a central region of a bone structure.

The present invention provides for vertebral fracture stabilization, as well as precise placement of the curable substance into a central region of a bone structure through a unipedicular approach.

One primary advantage of the present invention is its ability to create a central cavity in a vertebral body (or to centrally deliver bone cement) through a unipedicular approach.

The present invention is a curved needle having increased rigidity in its flexible end when the end is disposed in its straight position. This increased rigidity is due to a novel cable tensioning mechanism and to a segmented tube design (as opposed to the conventional longitudinally-slotted tube design).

The curved needle of the present invention also displays decreased fatigue stress in the flexible end during bending (in comparison to slotted tube designs) because no component of the tube of the present invention is internally bent. This is due to use of a cable and separate, nested tube segments in the present invention. Therefore, the present invention displays an increased durability of the flexible tubular end. Lastly, the present invention provides for increased control. This increased control is due to its slow curve progression brought about by providing a high number of driving screw turns, that is, a low pitch.

In preferred embodiments, the steerable needle has a unique driving mechanism that comprises a) a driving screw that includes both left-hand and right-hand threads, and b) cable couplings that slide in opposite directions, pulling and releasing the cable. The advantages provided by the two couplings that slide in opposite directions are the constant tension of the cables, and the rigidity and stability of the curved end.

Therefore, in accordance with the present invention, there is provided a steerable needle comprising:
i) a tube having a rigid proximal end portion and a flexible distal end portion,
ii) a cable having a proximal end portion and a distal end portion, the distal end portion of the cable being attached to the flexible distal end portion of the tube, and
iii) a drive mechanism comprising :
   a) a driving screw comprising a proximal thread and a distal thread, wherein the proximal thread has a first direction, the distal thread has a second direction, and the first direction is opposite the second direction, and
   b) first and second cable couplings adapted to slide in opposite directions, pulling and releasing the cable.
iv) a proximal handle connected to the rigid proximal portion of the tube, the handle comprising an actuator connected to the proximal end portion of the cable for tensioning the cable.

In other preferred embodiments, the steerable needle comprises a cable-tensioning means.

In some embodiments, the steerable needle possesses a pre-tensioned cable. The advantage of the pre-tensioned cable is that, when used with a particular tube design, it forces the tube to a straight configuration, and so provides rigidity in the flexible distal portion of the straightened tube.

Therefore, in accordance with the present invention, there is provided a steerable needle comprising:
i) a tube having a rigid proximal end portion and a flexible distal end portion,
ii) a cable attached to the flexible distal end portion of the tube,
iii) a proximal handle connected to the rigid proximal portion of the tube, the handle comprising an actuator connected to the cable for tensioning the cable,
   wherein the cable is under tension, and
   wherein the flexible distal end portion of the tube is substantially straight.

Generally, the flexible end of the tube comprises a column of nested segments. The separate nature of these segments allows for the overall bending of the tube end without requiring any bending within any single segment. Thus, the separate nature of the nesting segments provides an increased flexural fatigue strength of the device. In some embodiments, each end of each nested cylindrical segment is flat. Accordingly, tensioning of the cable associated with these segments produces a compression of this column of segments, thereby providing rigidity to the flexible tube end in its straight configuration.

Therefore, in accordance with the present invention, there is provided a steerable needle comprising:
i) a tube having a rigid proximal end portion and a flexible distal end portion,
ii) a cable running along (and preferably within) the tube and having a proximal end portion and a distal end portion, the distal end portion of the cable being attached to the flexible distal end portion of the tube,
iii) a proximal handle connected to the rigid proximal portion of the tube, the handle comprising an actuator connected to the cable for tensioning the cable,
   wherein the flexible distal end portion of the tube comprises a plurality of nested, separate tubular segments.

Some embodiments of the present invention are characterized by an ease of manual control. In these embodiments, motion of the flexible tube end is controlled by the controlled movement of the cable couplings, which is driven by a high number of the driving screw turns. Such control can also be attained by predetermining the pitch of the screw. Preferably, the screw thread has a pitch of between 1 mm and 2 mm. If the pitch is smaller than 1 mm, then an excessive number of turns is required to obtain appropriate curvature of the flexible distal end portion of the device. If the pitch is greater than 2 mm, the user has substantially less manual control over the device.

Therefore, in accordance with the present invention, there is provided a steerable needle comprising:
i) a tube having a rigid proximal end portion and a flexible distal end portion,
ii) a cable attached to the flexible distal end portion of the tube,
iii) a drive mechanism comprising :
   a) a driving screw comprising a proximal thread and a distal thread, wherein the proximal thread has a first direction, the distal thread has a second direction,
   b) first and second cable couplings adapted to slide in opposite directions, pulling and releasing the cable,
iv) a proximal handle connected to the rigid proximal portion of the tube, the handle comprising an actuator connected to the cable for tensioning the cable,
   wherein the proximal thread has a pitch of between 1 mm and 2 mm.

### DESCRIPTION OF THE FIGURES

FIG. 1 discloses a curved needle assembly.

FIG. 2 discloses a driving mechanism.

FIG. 3 discloses a driving mechanism displayed without a cover.

FIG. 4 illustrates the flexible distal end portion of the steerable needle.

FIGS. 5a and 5b disclose distal tip segments.

FIG. 6 discloses an outer shell component of FIG. 5a.

FIG. 7 discloses an insert 19 component of FIG. 5a.

FIG. 8 illustrates an intermediate nesting segment.

FIG. 9 illustrates a proximal nesting segment of the flex.

FIG. 10 discloses a hypodermic tube assembly.

FIG. 11 illustrates an axial cross-section of a funnel component.

FIG. 12 illustrates one half-shell of handle insert component.

FIG. 13 illustrates a driving screw component.

FIG. 14 illustrates a cable coupling component.

FIGS. 15, 16 and 17 illustrate the various parts of the cable clamping and tensioning mechanism.

FIGS. 18a and 18b illustrate views of the left cover component of the handle.

FIGS. 19a and 19b illustrate the right cover of the handle.

### DETAILED DESCRIPTION OF THE INVENTION

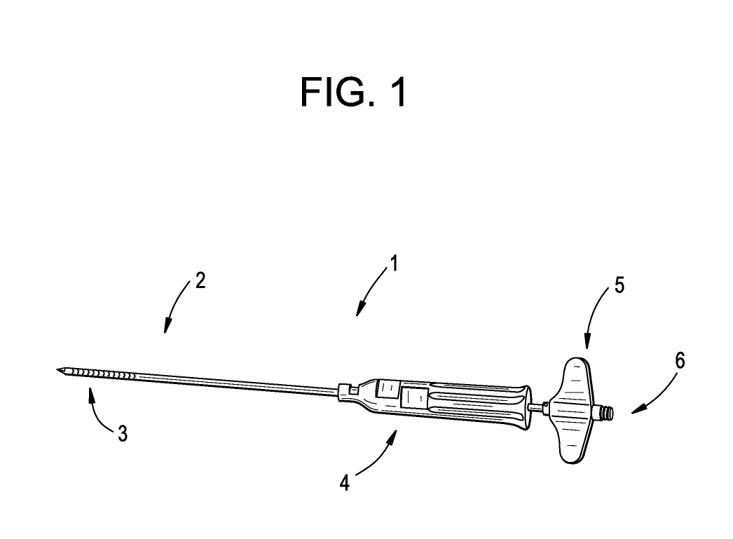

Now referring to FIG. 1, there is provided a curved needle assembly 1. The instrument comprising: tube assembly 2, shrink tubing 3, handle 4, driving handle 5, and luer fitting 6.

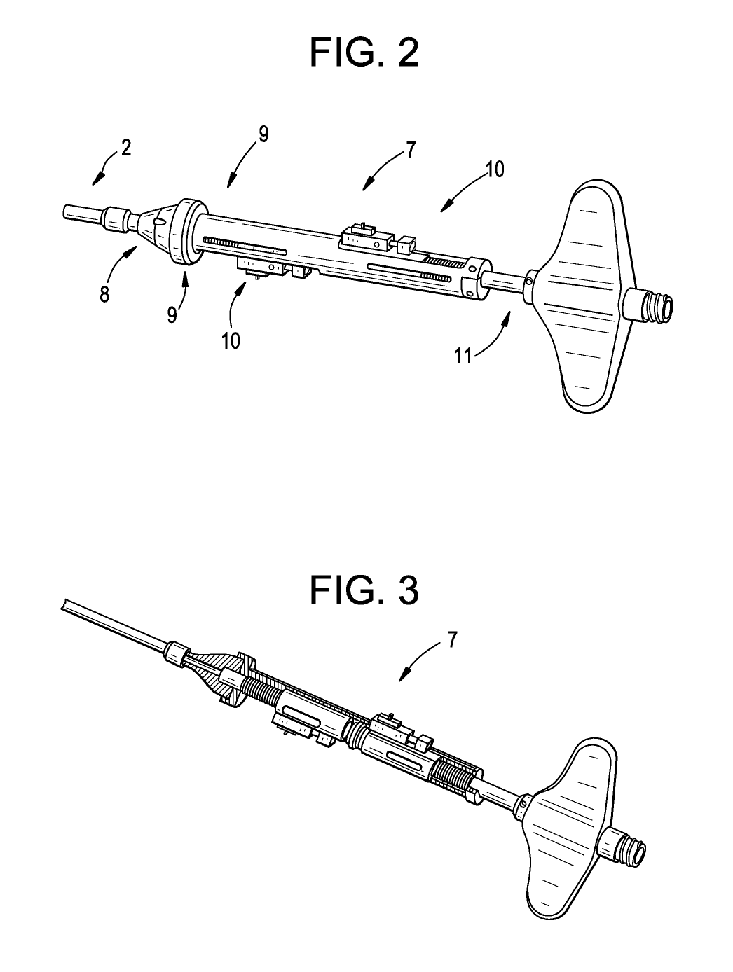

Now referring to FIG. 2, the driving mechanism 7 comprises a split wire funnel 8, split handle inserts 9, left and right cable couplings 10 and driving shaft 11.

FIG. 3 shows the driving mechanism without a cover.

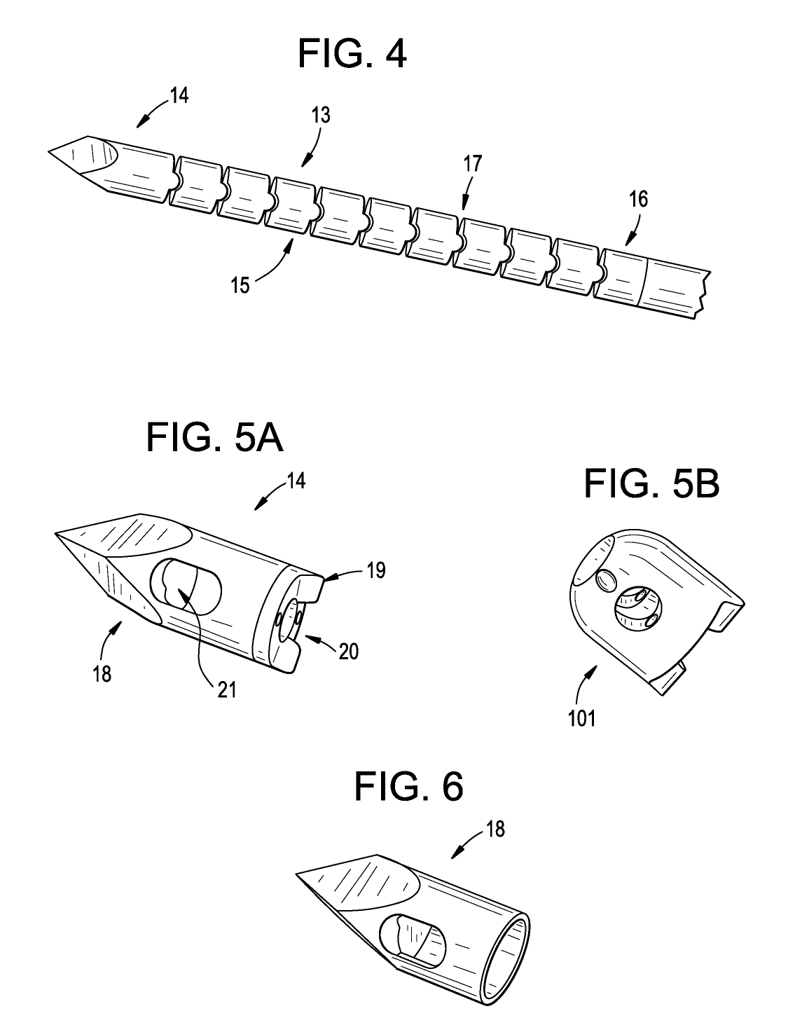

FIG. 4 illustrates the flexible distal end portion of the steerable needle 13, which comprises a top segment 14, intermediate segments 15 and bottom segment 16. The flexible portion acts via unidirectional action, as adjacent segments define a gap 17 therebetween. During actuation of the flex, these gaps close to produce the concave side of the flex.

FIG. 5a discloses a distal tip segment 14 comprising outer shell 18, pressed or welded insert 19, central hole 20 and a side hole for injecting cement 21. FIG. 5b discloses one preferred distal tip segment 101 having an integral construction.

FIG. 6 shows the outer shell 18 component of FIG. 5a.

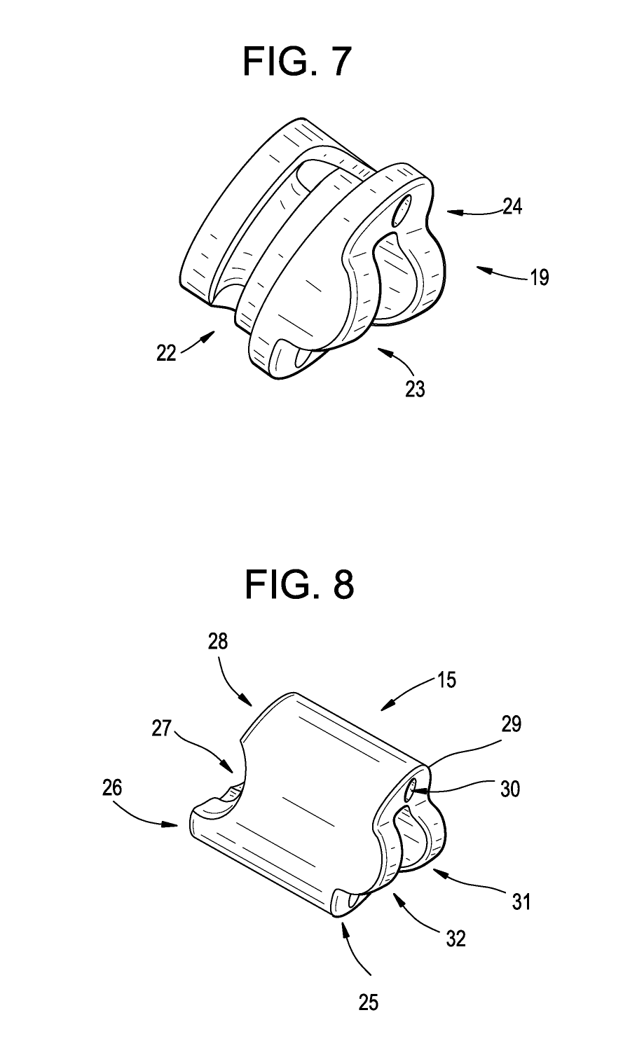

FIG. 7 shows the insert 19 component of FIG. 5a. The insert includes a groove 22 in its generally cylindrical body for the cable fixation, two small holes 24 for the cable insertion, and two radiused protrusions 23 for nested connection, alignment and pivoting relative to its adjacent intermediate segment 15.

FIG. 8 illustrates intermediate nesting segment 15 comprising horizontal surfaces 25 and 26 and angled surfaces 28 and 29, small holes for the cable insertion 30, central hole 31 for cement injection, radiused protrusion 32 and cavity 27.

Generally, an intermediate nesting segment comprises a distal end having one of a radiused projection and a radiused recess, and a proximal end having the other of the radiused projection and the radiused recess. In some embodiments, at least one of the nested, separate segments has a generally cylindrical shape defining a longitudinal axis, and wherein the radiused projection and radiused recess are each provided on a line parallel to the longitudinal axis. These conditions allow for linear nesting along one surface of the flexible portion of the needle.

In preferred embodiments, the distal end of the intermediate segment 15 further has a flat surface, its corresponding proximal end further has a flat surface, and the radiused projection and radiused recess are each provided on a line substantially parallel to the longitudinal axis.

In some nesting arrangements, the flexible portion of the needle comprises a first nesting segment and a second nesting segment adjacent the first nesting segment, wherein the first nesting segment comprises a projection, the second nesting projection comprises a recess, wherein the projection is nested in the recess, and wherein the first and second nesting segments define a gap therebetween.

In some embodiments, a first intermediate segment comprises a generally cylindrical shape having a first end having first and second projections extending therefrom, and a second end comprising first and second recesses therein. In some embodiments, the first and second projections define first and second end surfaces therebetween, the first and second recesses define third and fourth surfaces therebetween, wherein the first and third surfaces are parallel and the second and fourth surfaces are skewed. In embodiments producing the gap, the second and fourth surfaces are oriented towards each other.

In some embodiments, the projections define a first radius, the recesses define a second radius, and the first radius is substantially equal to the second radius. This allows for a high degree of nesting.

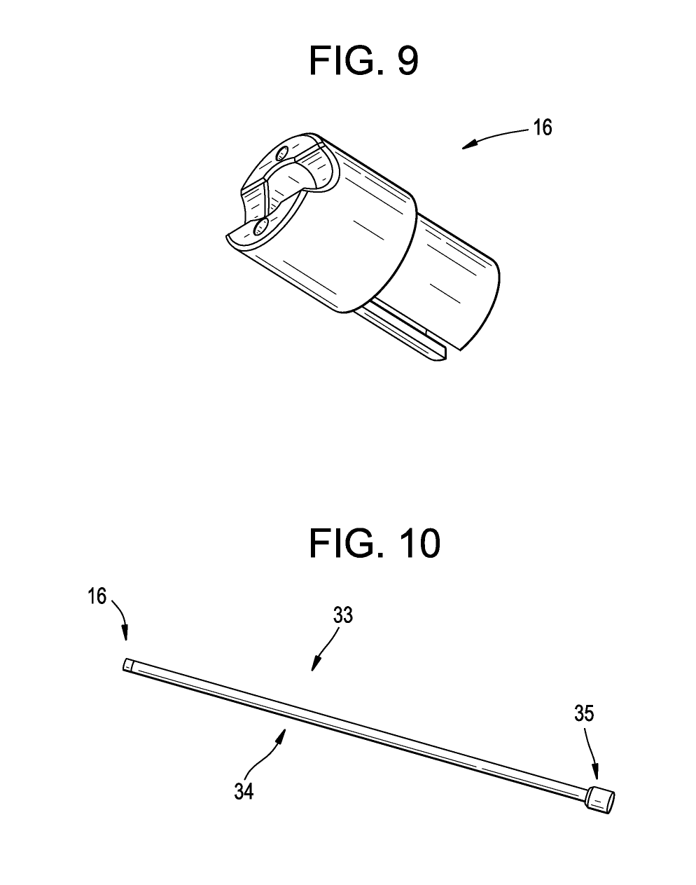

FIG. 9 illustrates the proximal segment 16 of the flex. It has a substantially cylindrical body, a pair of distal recesses, but no proximal projections. This segment 16 may be welded to the hypodermic tubing 34 (which is shown in FIG. 10) that is proximal thereto within the device.

FIG. 10 shows the hypodermic tube assembly 33 comprising the bottom segment 16, the hypodermic tube 34, and the coupling 35 welded to the tube 34.

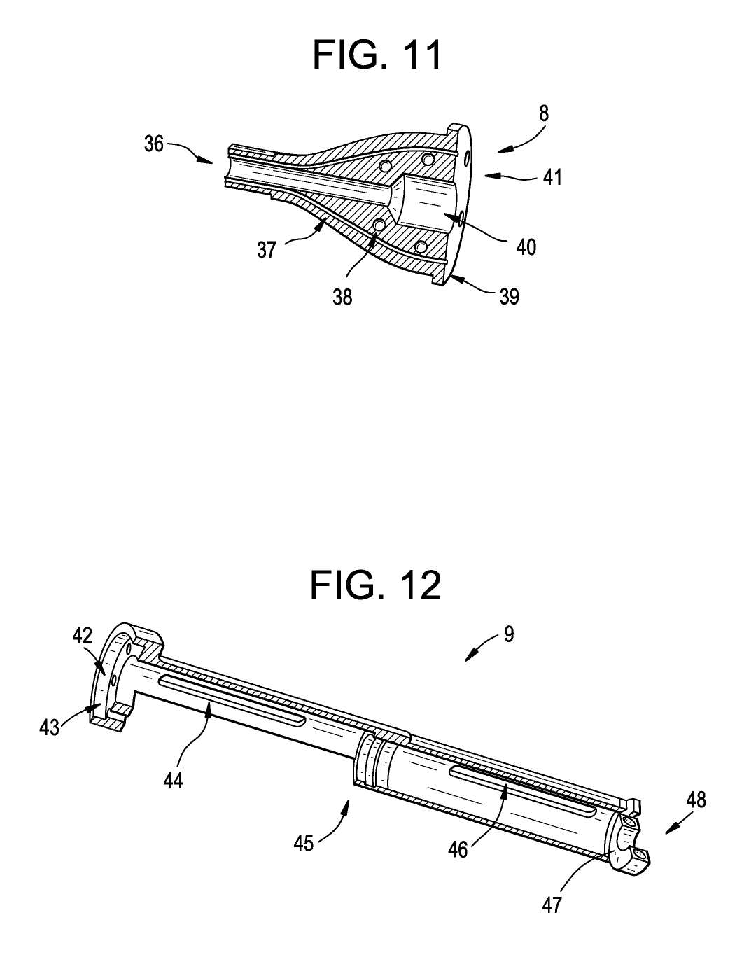

FIG. 11 illustrates an axial cross-section of funnel 8, which includes a hole 36 for cement delivery, a hole 40 for aligning the driving shaft 11, channels 37 for receiving the cable, a flange 39 for assembly with handle inserts 9, holes 38 for receiving the pins that hold the funnel together, and holes 41 for attaching the funnel to the handle insert cover 9.

FIG. 12 illustrates one half-shell of handle insert 9 comprising groove 43 for funnel assembly, holes 42 for attaching inserts to the funnel by pins, slots 44 and 46 for guiding the cable couplings 10, the groove 45 for aligning with the flange of driving screw 11, hole 47 for guiding the driving screw 11, and holes 48 for assembly with the other half-shell of the handle insert using pins.

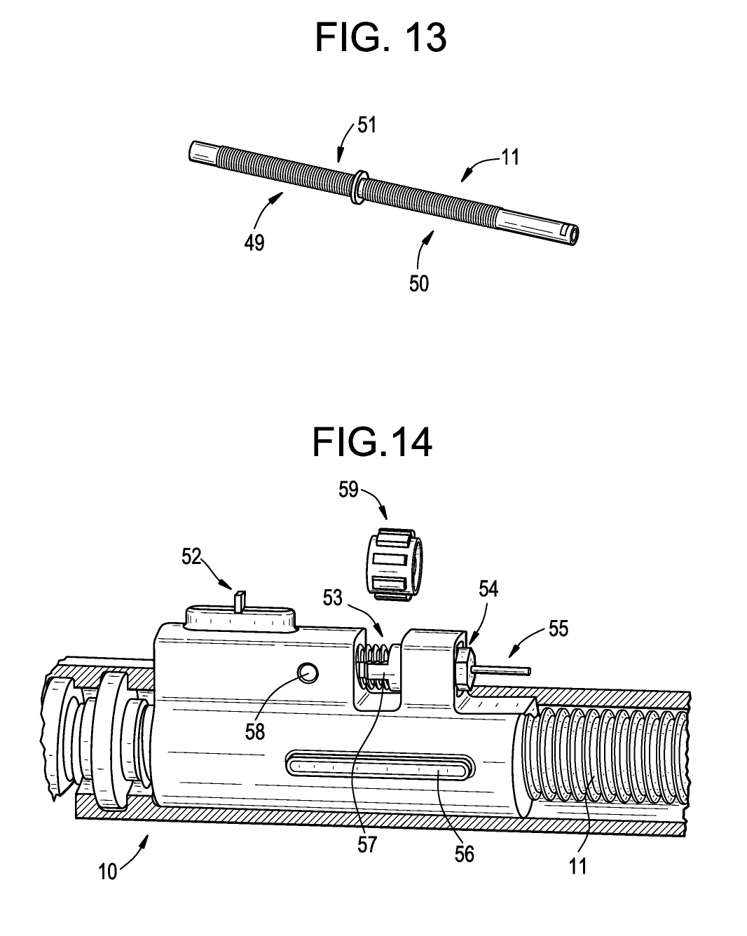

FIG. 13 illustrates the driving screw 11 comprising left 49 and right 50 threads, and an intermediate flange 51 for aligning the screw with handle inserts 9.

FIG. 14 illustrates cable coupling 10 comprising indicator of the flex angle 52, tensioning screw 53, male clamp 54, alignment guide 56, female clamp 57, pin 58, and nut 59. Cable 55 is fed into male clamp 54 to secure the cable.

In general, the cable tensioning mechanism works as follows: The cable is threaded through segments 14-16, tube assembly 33, funnel 8 and screw 53. The cable ends are held by the female clamp 57 and male clamp 54 of the two cable couplings 10. By turning nut 59, the screw 53 of the two cable couplings 10 is pulled back, thereby tensioning both ends of the cable.

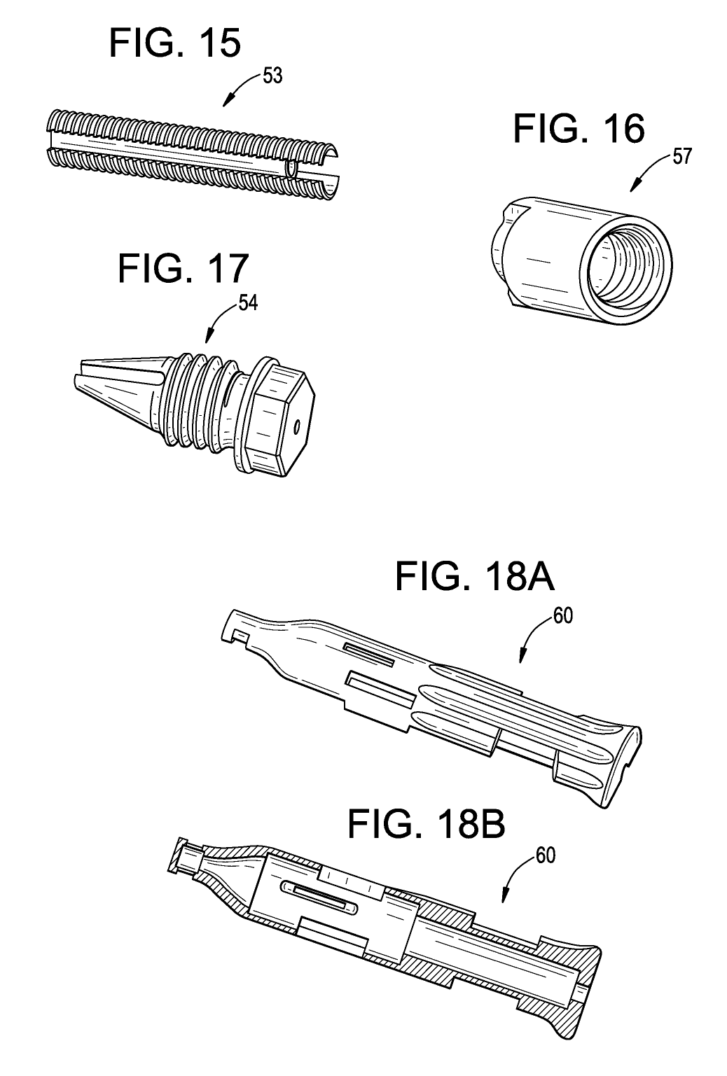

FIGS. 15, 16 and 17 illustrate the various parts of the cable clamping and tensioning mechanism. FIG. 15 discloses one preferred tensioning screw component 53 of the cable coupling. This screw component 53 has a thread thereon. FIG. 16 discloses one preferred female-threaded clamp component 57 of the cable coupling. FIG. 17 discloses one preferred male-threaded clamp component 54 of the cable coupling.

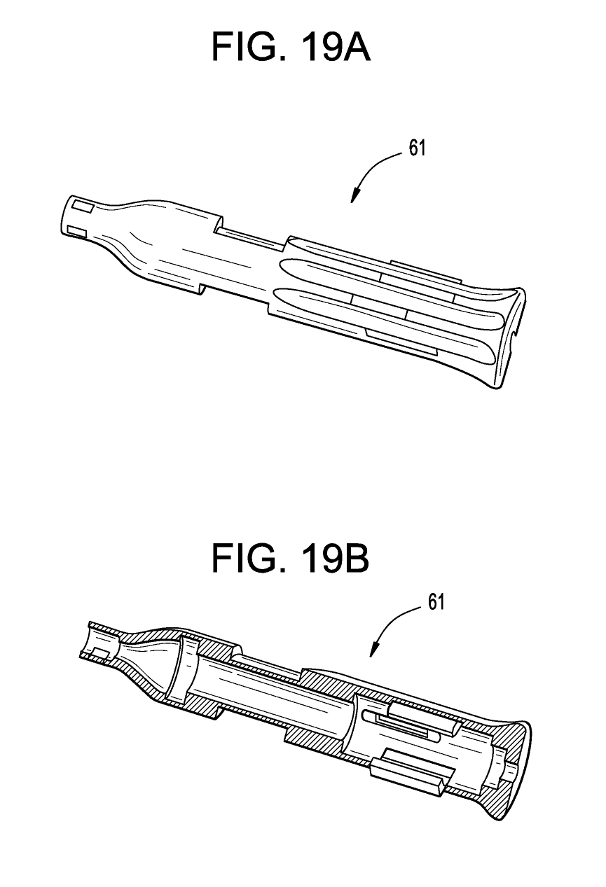

FIGS. 18a and 18b illustrate views of the left cover 60 of the handle 4, while FIGS. 19a and 19b illustrate the right cover 61 of handle 4.

To assemble the device, the cable 55 is threaded through the hole 24 of the insert 19, looped through the groove 22 and threaded back into hole 24. The cable is then locked by the outer shell 18 assembly with the insert 19. The middle segments 15 are then threaded into the cable with their horizontal surfaces 25 facing the same direction. The flexible end 13 is then assembled with the tube assembly 33 and the cable ends are threaded through the tube. The left and right couplings 10 are assembled onto the driving shaft 11 and then the assembly is surrounded with the handle inserts 9. The funnel 8 is assembled with handle inserts 9. The cable ends are then guided into the funnel channels 37 and the hole of the tensioning screw 53. The tube assembly 2 is then assembled with the funnel 8 and cables are pre-tensioned by hand.

The cable ends are locked by the male clamp 54. The final tensioning of the cable is achieved by turning the nuts. When this is done, the covers 60 and 61 are placed around the assembly and locked. The shrink tubing 3 is placed over the flexible end to prevent cement leakage.

The flexing of the distal flexible end of the device is achieved by rotating the driving handle 5. This rotation in turn turns the driving shaft 11, and cable couplings 10 slide in opposite directions—pulling and releasing the cable. As the cable is fixed at the tip segment, it slides through the segments, either flexing or straightening the flexible end of the device.

The present invention can be practiced through a unipedicular approach as well as a bipedicular approach.

To inject cement into the vertebral body, an injection system having a cement reservoir containing flowable cement is attached to the luer 6 and cement is flowed through the central hole of the driving screw, funnel and the tube assembly into the flexible end of the device, and is finally ejected through the side hole 21 and into the vertebral body.

In the embodiments shown, the device ejects cement through a hole in the sidewall of the tube. However, in other embodiments, the cement may be axially ejected via an endhole opening through a distal end portion of the tube.

Preferred bone pastes include bone cements (such as acrylic-based bone cements, such as PMMA-based bone cements), pastes comprising bone particles (either mineralized or demineralized or both; and either autologous, allogenic or both), and ceramic-based bone cements (such as HA and TCP-based pastes).

In some embodiments, the flexible needle of the present invention may also be used as a conduit for cement delivery. In some embodiments thereof, the proximal end portion of the tube is fluidly connected to a cement reservoir.

We claim:

1. A steerable needle comprising:
i) a tube having a rigid proximal end portion and a flexible distal end portion,
ii) a cable having a proximal end portion and a distal end portion, the distal end portion of the cable being attached to the flexible distal end portion of the tube, and
iii) a drive mechanism comprising:
a) a driving screw comprising a proximal thread and a distal thread, wherein the proximal thread has a first direction, the distal thread has a second direction, and the first direction is opposite the second direction, and
b) first and second cable couplings adapted to slide in opposite directions, pulling and releasing the cable.
iv) a proximal handle connected to the rigid proximal portion of the tube, the handle comprising an actuator connected to the proximal end portion of the cable for tensioning the cable.

2. The needle of claim 1 wherein the driving screw has a flange positioned between the proximal thread and the distal thread.

3. The needle of claim 1 wherein the proximal end portion of the tube is fluidly connected to a cement reservoir.

4. A steerable needle comprising:
i) a tube having a rigid proximal end portion and a flexible distal end portion,
ii) a cable attached to the flexible distal end portion of the tube,
iii) a drive mechanism comprising:
a) a driving screw comprising a proximal thread and a distal thread, wherein the proximal thread has a first direction, the distal thread has a second direction,
b) first and second cable couplings adapted to slide in opposite directions, pulling and releasing the cable,
iv) a proximal handle connected to the rigid proximal portion of the tube, the handle comprising an actuator connected to the cable for tensioning the cable,
wherein the proximal thread has a pitch of between 1 mm and 2 mm.

5. The needle of claim 4 wherein the proximal end portion of the tube is fluidly connected to a cement reservoir.

6. A steerable needle comprising:
i) a tube having a rigid proximal end portion and a flexible distal end portion,
ii) a cable attached to the flexible distal end portion of the tube,
iii) a proximal handle connected to the rigid proximal portion of the tube, the handle comprising an actuator connected to the cable for tensioning the cable,
wherein the cable is under tension, and
wherein the flexible distal end portion of the tube is substantially straight.

7. The steerable needle of claim 6 wherein the flexible distal end portion of the tube comprises a plurality of nested segments, each segment having a proximal end and a distal end.

8. The steerable needle of claim 7 wherein a first segment has a substantially flat proximal end.

9. The steerable needle of claim 8 wherein a second segment has a substantially flat distal end.

10. The steerable needle of claim 9 wherein the substantially flat proximal end of the first segment abuts the substantially flat distal end of the second segment.

11. A steerable needle comprising:
i) a tube having a rigid proximal end portion and a flexible distal end portion,
ii) a cable running along the tube and having a proximal end portion and a distal end portion, the distal end portion of the cable being attached to the flexible distal end portion of the tube,
iii) a proximal handle connected to the rigid proximal portion of the tube, the handle comprising an actuator connected to the cable for tensioning the cable,
wherein the flexible distal end portion of the tube comprises a plurality of nested, separate tubular segments.

12. The needle of claim 11 wherein at least one of the nested separate segments comprises a distal end having one of a radiused projection and a radiused recess, and a proximal end having the other of the radiused projection and the radiused recess.

13. The steerable needle of claim 12 wherein at least one of the nested, separate segments has a generally cylindrical shape defining a longitudinal axis, and wherein the radiused projection and radiused recess are each provided on a line parallel to the longitudinal axis.

14. The steerable needle of claim 12 wherein the distal end further has a flat surface, and the proximal end further has a flat surface, and wherein the radiused projection and radiused recess are each provided on a line parallel to the longitudinal axis.

15. The steerable needle of claim 12 comprising a first and a second nested segment adjacent the first nested segment, wherein the first nested segment comprises a projection, the second nested projection comprises a recess, wherein the projection is nested in the recess, and wherein the first and second nested segments define a gap therebetween.

16. The steerable needle of claim 12 wherein a first segment comprises a generally cylindrical shape having a first end having first and second projections extending therefrom, and a second end comprising first and second recesses therein.

17. The steerable needle of claim 16 wherein the first and second projections define first and second end surfaces therebetween, the first and second recesses define third and fourth surfaces therebetween, and wherein the first and third surfaces are parallel and the second and fourth surfaces are skewed.

18. The steerable needle of claim 17 wherein the second and fourth surfaces are oriented towards each other.

19. The steerable needle of claim 16 wherein the projections define a first radius, the recesses define a second radius, and the first radius is substantially equal to the second radius.

20. The steerable needle of claim 11 wherein the tube further comprises i) a sidewall; ii) a closed, distal tip and iii) a sidehole opening through a distal end portion of the sidewall.

21. The steerable needle of claim 11 wherein the tube further comprises i) an endhole opening through a distal end portion of the tube.

22. The needle of claim 11 wherein the cable runs within the tube.
